(19) United States
(12) Patent Application Publication
(10) Pub. No.: US 2003/0224357 A1
(43) Pub. Date: Dec. 4, 2003

(54) Method and system for predicting nucleic acid hybridization thermodynamics and computer-readable storage medium for use therein

(76) Inventors: John SantaLucia JR., Harper Woods, MI (US); Nicolas Peyret, San Mateo, CA (US)

Correspondence Address: BROOKS & KUSHMAN P.C., 22nd Floor, 1000 Town Center, Southfield, MI, 48075, US

(21) Appl. No.: 09/876,549

(22) Filed: Jun. 7, 2001

Related U.S. Application Data
(60) Provisional application No. 60/209,778, filed on Jun. 7, 2000.

(51) Int. Cl. C12Q 1/68; G06G 7/48; G06G 7/58

(52) U.S. Cl. 435/6; 703/11

(57) ABSTRACT

Method and system to predict and optimize probe-target hybridization are provided. The method may be implemented using six interactive, interrelated, software modules. Module 1 predicts the hybridization thermodynamics of a duplex given the two strands. Module 2 finds the best primer of a given length binding to a given target. Module 3 executes a primer walk to find alternative binding sites of a given primer on a given target. Module 5 is a combination of Modules 2 and 3. Module 6 finds the alternative binding sites of a given primer on a given target (Module 3) and calculates the concentration of target with primer bound at primary and alternative sites. Module 7 is a combination of Modules 2 and 5 and also calculates the various concentrations. The six modules can be operated either through an interactive user interface or using batch file submission as provided by Module 4. The program is suited to predict DNA/DNA, RNA/RNA, and RNA/DNA systems.

### CROSS-REFERENCE TO RELATED APPLICATION

This application claims the benefit of U.S. provisional application Serial No. 60/209,778, filed Jun. 7, 2000, entitled “Nucleic Acid Hybridization Prediction and Biochemical Techniques Utilizing Same.”

### STATEMENT REGARDING FEDERALLY SPONSORED RESEARCH OR DEVELOPMENT

This invention was made with government support under Grant No. HG02020 provided by NIH. The United States Government has certain rights in the invention.

### BACKGROUND OF THE INVENTION

1. Field of the Invention

This invention relates to methods and systems for predicting nucleic acid hybridization thermodynamics and computer-readable storage medium for use therein.

2. Background Art

Improvement of the efficiency of hybridization-based techniques requires the optimization of the binding between two sequences. Accurate prediction of the thermodynamics allows optimal choice of the sequences, temperature, and salt conditions. Hence, the prediction of nucleic acid thermodynamics is important to optimize techniques like PCR (Saiki et al., 1988), Southern and Northern blotting (Southern, 1975), antigene targeting (Freier, 1993), and Kunkel site-directed mutagenesis (Kunkel et al., 1987).

Hybridization prediction is also important for designing DNA microchips that have a wide field of application ranging from diagnostics (Hacia, 1999; Yershov et al., 1996) to gene expression analysis (Ferea et al., 1999) and drug discovery (Debouk and Goodfellow, 1999). Microchips contain a large number of DNA probe sequences that have to be designed to specifically hybridize target sequences in a pool of DNA fragments. First, a DNA probe should be designed to bind to only one site of only one DNA target. Second, the different DNA probe sequences need to hybridize to their targets under the same temperature and solution conditions. Moreover, in sequencing by hybridization (Fodor et al., 1993; Mirzabekov, 1994) where microchips are used to determine the sequence of given DNA, one has to be able to know hybridization thermodynamics to discriminate signals resulting from perfectly matched and mismatched probe/target hybridizations.

Another widely used technique that requires hybridization prediction is the fluorescence in situ hybridization (FISH) technique (Gall and Pardue, 1969). In this technique, a fluorescent tagged nucleic acid probe is designed to specifically hybridize cellular or tissue section nucleic acids. The target of these probes can either be endogenous DNA, messenger RNA or viral and bacterial sequences.

Therefore, FISH is used to monitor gene expression (McNicol and Farquharson, 1997), detect infectious agents (Bashir et al., 1994; McNicol and Farquharson, 1997; Pollanen et al., 1993), study cell cycle (McNicol and Farquharson, 1997), map chromosomes and study nuclear architecture (Heng et al., 1997). It was also determined that a set of probes can be used simultaneously (multiFISH) to detect different loci (Pagon, 1997). Once again, prediction of hybridization is essential to insure specificity. Nucleic-acid hybridization prediction is also important for the design of oligonucleotide aptamers or antisense oligonucleotides (Cohen, 1992) that can be used for various therapeutic applications. A new type of probes known as molecular beacons (Bonnet et al., 1999; Tyagi et al. 1998) that are very specific has been developed and shown to be efficient for mutation analysis (Giensendorf et al., 1998) and multiplex detection of single nucleotide variations (Marras et al., 1999). The design and prediction of the thermodynamics of these beacons is helped by hybridization thermodynamics prediction (Bonnet et al., 1999). Accurate prediction of hybridization is also important for the practical realization of DNA-based or more generally nucleic acid-based computers. (Adleman, L. M., 1994).

The development of molecular biology techniques based on hybridization (PCR, FISH, DNA microchips, etc.) has resulted in a need for efficient automated ways to design probes and primers. In the last decade, numerous algorithms have been developed to optimize the design of primers and probes for various applications (Rychlik and Rhoads, 1989)(Breslauer et al., 1986; Chen and Zhu, 1997; Dopazo et al., 1993; Haas et al., 1998; Hillier and Green, 1991; Hyndman et al., 1996; Li et al., 1997; Link et al., 1997; Pesole et al., 1998; Proutski and Holmes, 1996). Numerous unpublished software to predict primers are also made available by research groups and biotech companies on the World Wide Web (Primer3 from the Whitehead Institute for Biomedical Research. Primer Express™ from PE Biosystems, DNAstar from IDT, etc.)

There are currently many software packages on the market for DNA primer design including: OLIGO, PRIMER PREMIER, OSP, GCG, PrimerMaster, and Primo. None of the current programs, however, were written by experts in DNA thermodynamics; thus, there are many improvements that can be made. Nearly all of the current software packages contain mistakes that result from a lack of understanding of the underlying theory of DNA hybridization. PCR is a fairly robust process and thus even crude programs make predictions that work 90-95% of the time. Multiplex PCR primer design, however, is not at all trivial and detailed knowledge of the physical chemistry of DNA hybridization, and the availability of an accurate thermodynamic database are essential to reliable design of multiplex PCR primers. In multiplex PCR, several primers must be designed to specifically bind to different sites on target DNA at a given temperature with minimal background binding to mismatch sites and with minimal cross-hybridizations between pairs of primers. The design of molecular beacons for DNA oligonucleotide arrays is also very challenging because of the complex competing equilibria.

Most of the existing programs aiming at finding an optimum probe that binds a specific location on a target, however, do not include accurate stability rules for hybridization and neglect or poorly approximate competitive binding sites, strand folding and strand dimerization.

U.S. Pat. Nos. 5,593,834 and 6,027,884 to Lane et al. disclose methods to design and construct DNA sequences with selected reaction attributes.

In summary, prediction of nucleic acids thermodynamics is important to optimize various molecular biology techniques including multiplex PCR, DNA microchips, molecular beacons, and fluorescence in situ hybridization. Most of the available programs for probe design do not include a complete parameterization and often do not account for mismatches. Moreover, single strand folding is not taken into account, which often leads to inaccurate predictions.

### SUMMARY OF THE INVENTION

An object of the invention is to provide a method and system for predicting nucleic acid hybridization thermodynamics and computer-readable storage medium for use therein wherein the invention utilizes a thermodynamically rigorous approach to evaluate the quality of probes and simulate probe/target hybridization.

Another object of the invention is to provide a method and system for predicting nucleic acid hybridization thermodynamics and computer-readable storage medium for use therein wherein the invention also takes into account single strand folding thermodynamics to calculate effective hybridization thermodynamics.

In carrying out the above objects and other objects of the present invention, a method for predicting nucleic acid hybridization thermodynamics is provided. The method includes providing a database of thermodynamic parameters, receiving hybridization information which represents at least one sequence, receiving correction data, receiving a first set of data which represents hybridization conditions, and calculating hybridization thermodynamics including net hybridization thermodynamics based on the hybridization information, the thermodynamic parameters, the correction data and the first set of data.

The hybridization thermodynamics of individual single stranded, bimolecular and higher order complexes may be statistically weighted in a numerical process and the equilibrium concentration of each species is output.

The correction data may include folding correction data and/or linear correction data.

The thermodynamic parameters may include DNA thermodynamic parameters.

The DNA thermodynamic parameters may include dangling end parameters and/or coaxial stacking parameters.

The DNA thermodynamic parameters may further include terminal mismatch parameters.

The thermodynamic parameters may include RNA thermodynamic parameters and/or hybrid DNA/RNA thermodynamic parameters.

The thermodynamic parameters may further include DNA loop thermodynamic parameters.

The hybridization information may represent top and bottom strand sequences which form a duplex and wherein the hybridization thermodynamics are calculated for the duplex.

The hybridization information may further represent at least a section of a target and a length of at least one primer or probe complimentary to the target.

The hybridization thermodynamics may be calculated for a plurality of primers or probes complimentary to the target.

The hybridization information may represents at least a section of a target and a primer or probe.

A length of the target may be longer than a length of the primer or probe and wherein the hybridization thermodynamics are calculated for a best target/primer or target/probe complex and for competitive mismatch complexes.

Hybridization information may represent at least a section of a target and a primer or probe and wherein a length of a target is longer than the length of the primer or probe and wherein the hybridization thermodynamics are calculated for a best target/primer or target/probe complex and for competitive target/primer or target/probe complexes.

The method may further include calculating concentration of each species in a solution at a plurality of temperatures.

Hybridization information may also represent a primer or probe and wherein the length of the target is longer than a length of the primer or probe and wherein the hybridization thermodynamics are calculated for a best target/primer or target/probe complex and for competitive mismatch complexes and wherein the method may further comprise calculating concentration of every species in a solution at a plurality of temperatures.

The hybridization thermodynamics may be calculated for at least two best target/primer or target/probe complexes and for their corresponding competitive mismatch complexes and wherein the method may further comprise correcting for any interactions between the at least two best target/primer or target/probe complexes and their components.

Further in carrying out the above objects and other objects of the present invention, a system for predicting nucleic acid hybridization thermodynamics is provided. The system includes a database of thermodynamics parameters, means for receiving hybridization information which represents at least one sequence, and means for receiving correction data. The system further includes receiving a first set of data which represents hybridization conditions, and means for calculating hybridization thermodynamics including net hybridization thermodynamics based on the hybridization information, the thermodynamic parameters, the correction data and the first set of data.

The hybridization thermodynamics of individual single stranded, bimolecular and higher order complexes may be statistically weighted in a numerical process and the equilibrium concentration of each species is output.

The correction data may include folding correction data and/or linear correction data.

The thermodynamic parameters may include DNA thermodynamic parameters such as dangling end parameters.

The DNA thermodynamic parameters may include coaxial stacking parameters and/or terminal mismatch parameters.

The thermodynamic parameters may include RNA thermodynamic parameters and/or hybrid DNA/RNA thermodynamic parameters.

The thermodynamic parameters may further include DNA loop thermodynamic parameters.

The hybridization information may represent top and bottom strand sequences which form a duplex and wherein the hybridization thermodynamics are calculated for the duplex.

The hybridization information may also represent at least a section of a target and a length of at least one primer or probe complimentary to the target.

The hybridization thermodynamics may be calculated for a plurality of primers or probes complimentary to the target.

The hybridization information may represent at least a section of a target and a primer or probe.

A length of the target may be longer than a length of the primer or probe and wherein the hybridization thermodynamics are calculated for a best target/primer or target/probe complex and for competitive mismatch complexes.

Hybridization information may represent at least a section of a target and a primer or probe and wherein a length of a target is longer than the length of the primer or probe and wherein the hybridization thermodynamics are calculated for a best target/primer or target/probe complex and for competitive target/primer or target/probe complexes.

The system may further include means for calculating concentration of each species in a solution at a plurality of temperatures.

Hybridization information may also represent a primer or probe and wherein the length of the target is longer than a length of the primer or probe and wherein the hybridization thermodynamics are calculated for a best target/primer or target/probe complex and for competitive mismatch complexes and wherein the system may further comprise means for calculating concentration of every species in a solution at a plurality of temperatures.

The hybridization thermodynamics may be calculated for at least two best target/primer or target/probe complexes and for their corresponding competitive mismatch complexes and wherein the system may further comprise means for correcting for any interactions between the at least two best target/primer or target/probe complexes and their components.

Still further in carrying out the above objects and other objects of the present invention, a computer-readable storage medium having stored therein a database of thermodynamics parameters and a computer program are provided. The computer program executes the steps of: a) receiving hybridization information which represents at least one sequence; b) receiving correction data; c) receiving a first set of data which represents hybridization conditions; and d) calculating hybridization thermodynamics based including net hybridization thermodynamics based on the hybridization information, the thermodynamic parameters, the correction data and the first set of data.

The hybridization thermodynamics of individual single stranded, bimolecular and higher order complexes may be statistically weighted in a numerical process and the equilibrium concentration of each species is output.

The correction data may include folding correction data and/or linear correction data.

The thermodynamic parameters may include DNA thermodynamic parameters.

The DNA thermodynamic parameters may include dangling end parameters and/or coaxial stacking parameters.

The DNA thermodynamic parameters may further include terminal mismatch parameters.

The thermodynamic parameters may include RNA thermodynamic parameters and/or hybrid DNA/RNA thermodynamic parameters.

The thermodynamic parameters may further include DNA loop thermodynamic parameters.

The hybridization information may represent top and bottom strand sequences which form a duplex and wherein the hybridization thermodynamics are calculated for the duplex.

The hybridization information may represent at least a section of a target and a length of at least one primer or probe complimentary to the target.

The hybridization thermodynamics may be calculated for a plurality of primers or probes complimentary to the target.

The hybridization information may represent at least a section of a target and a primer or probe.

A length of the target may be longer than a length of the primer or probe and wherein the hybridization thermodynamics are calculated for a best target/primer or target/probe complex and for competitive mismatch complexes.

Hybridization information may represent at least a section of a target and a primer or probe and wherein a length of a target is longer than the length of the primer or probe and wherein the hybridization thermodynamics are calculated for a best target/primer or target/probe complex and for competitive target/primer or target/probe complexes.

The program may further execute the step of calculating concentration of each species in a solution at a plurality of temperatures.

Hybridization information may also represent a primer or probe and wherein the length of the target is longer than a length of the primer or probe and wherein the hybridization thermodynamics are calculated for a best target/primer or target/probe complex and for competitive mismatch complexes and wherein the program may execute the step of calculating concentration of every species in a solution at a plurality of temperatures.

The hybridization thermodynamics may be calculated for at least two best target/primer or target/probe complexes and for their corresponding competitive mismatch complexes and wherein the program may execute the step of correcting for any interactions between the at least two best target/primer or target/probe complexes and their components.

The above objects and other objects, features, and advantages of the present invention are readily apparent from the following detailed description of the best mode for carrying out the invention when taken in connection with the accompanying drawings.

### BRIEF DESCRIPTION OF THE DRAWINGS

FIG. 1 is a schematic drawing wherein multiple equilibria are considered for concentration calculations;

FIG. 2a is a schematic drawing of a user input interface wherein the user provides various input information for a first module of the invention;

FIG. 2b is a schematic drawing of a user output interface wherein a computer provides output information corresponding to the input information of FIG. 2a;

FIG. 3a is a schematic drawing of a user input interface wherein the user provides various input information for a second module of the invention;

FIG. 3b is a schematic drawing of a user output interface wherein a computer provides output information corresponding to the input information of FIG. 3a;

FIG. 4a is a schematic drawing of a user input interface wherein the user provides various input information for a third module of the invention;

FIG. 4b is a schematic drawing of a user output interface wherein a computer provides output information corresponding to the input information of FIG. 4a;

FIG. 5a is a schematic drawing of a user input interface wherein the user provides various input information for a fifth module of the invention;

FIG. 5b is a schematic drawing of a user output interface wherein a computer provides output information corresponding to the input information of FIG. 5a;

FIG. 6 is a block diagram flow chart illustrating the solution of conservation equations of the present invention;

FIG. 7 is a schematic diagram illustrating multiplex PCR design;

FIG. 8 shows prediction of molecular beacon net hybridization thermodynamics;

FIG. 9 shows simulation of molecular beacon hybridization concentrations at temperatures from 0 to 100° C.;

FIG. 10 is a diagram of match vs. mismatch hybridization;

FIG. 11 shows match vs. mismatch hybridization simulation at different temperatures;

FIG. 12 shows a general case of competitive hybridization equilibria that can be solved using the described numerical methods; and

FIG. 13 is an example of simultaneous equations for the general five molecule case.

### DETAILED DESCRIPTION OF THE PREFERRED EMBODIMENTS

In general, the method and system of the present invention include rigorous thermodynamic parameterization for Watson-Crick base pairs, internal mismatches, terminal mismatches, terminal dangling ends, co-axial stacking interactions, sodium and magnesium salt dependence, denaturants (urea, formamide, DMSO). In addition, loop parameters for hairpins, internal loops, bulges, and multibranched loops are included. For DNA essentially all the parameters have been previously published or all included in the Appendix hereto. Specifically, the parameters which have been published include Watson-Crick parameters, sodium dependence, GT, GA, CT, AC, AA, CC, GG, and TT mismatches. The parameters included herein include dangling ends, terminal mismatches, DNA loop parameters, and co-axial stacking parameters. For RNA, the parameters have been published by Douglas H. Turner et al. For DNA/RNA hybrid duplexes, the parameters have been published by Naoki Sugimoto.

The method and system are adapted for future implementation of parameters for modified nucleosides (including but not limited to inosine, 5-nitroindole, PNA, MOE-modified RNA, and iso-bases). With these parameters, it is possible to predict the melting temperature, Tm, of a duplex within 2° C. on average. Correction for surface effects for DNA chip arrays is also implemented. In addition to predicting duplex hybridization, the software accounts for single-strand secondary structure. This is accomplished by a new numerical procedure for solving complex coupled equilibria (multi-state model). With this approach, it is possible to accurately predict not only the Tm for hybridization but also the concentration of every species in the solution (e.g. match duplex, mismatch, duplex, folded target, folded primer, primer dimer, etc.) at every temperature from 0 to 100° C. Thus, it is possible to use this software to design oligonucleotide hybridization with optimized temperature, salt, and strand concentrations.

Predicting Accurately Primer Target Interaction Stability

The stability of a primer/target or probe/target complex can be described by the free energy of association of the probe and the target. The most accurate way to calculate free energy of association is to use the nearest-neighbor model with accurate thermodynamic parameters. Thermodynamic parameters should account for Watson-Crick base pairs (SantaLucia et al., 1996; Allawi & SantaLucia, 1997; SantaLucia, 1998), single mismatches (Allawi & SantaLucia, 1997), terminal mismatches (disclosed herein), dangling ends (Bonmmarito, Pugret & SantaLucia, 2000) and possibly double mismatches. Proper calculation of the monovalent and divalent salt dependence is also important (SantaLucia, 1998). Other loop motifs for hairpins, bulge, internal loops and multi-branched loops are important for single strand secondary structure prediction, but are often very crudely approximated. Moreover, when primer and target folding can occur, a set of coupled equilibria should be used to model the system. The nearest-neighbor model needs to be used to determine the equilibrium constant of each equilibrium. The determination of possible primer or target folding can be addressed by using secondary-structure prediction algorithms like M. Zuker's MFOLD (Zuker, 1989).

Secondary Structure and Net Hybridization Thermodynamics

Species Concentration Calculations

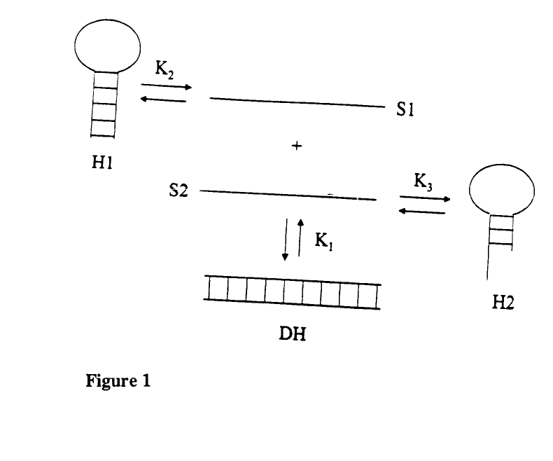

Consider a system of strands S1 and S2 with four states: folded target, folded probe, probe bound to target, and random coil target and probe. The model can be described by three equilibria as shown in FIG. 1.

The concentrations of every species for such a system can be analytically determined. The three equilibrium constants for such a system are shown below:

$$\begin{matrix}S1 + S2 \overleftarrow{\to } DH {K}_{1} = \frac{[ DH ]}{[ S1 ] [ S2 ]} & ( 1 ) \\ S1 \overleftarrow{\to } H1 {K}_{2} = \frac{[ H1 ]}{[ S1 ]} & ( 2 ) \\ S2 \overleftarrow{\to } H2 {K}_{3} = \frac{[ H2 ]}{[ S2 ]} & ( 3 )\end{matrix}$$

where S1, S2, H1, H2, and DH are the random coil S1, the random coil S2, the folded strand H1, the folded strand H2, and the double helix DH, respectively. The conservation of S1 and S2 leads to the following equations:

C_{S1}^{Total}=S1+H1+DH (4)

C_{S2}^{Total}=S2+H2+DH (5)

where C_{S1}^{Total} are the total concentrations of S1 and S2. [DH] and [S2] can be expressed as a function of [S1] by substituting the [H1] obtained from Equation 2, in Equation 6, and then substituting the [DH] obtained by Equation 6 in Equation 1.

[DH]=C_{S1}^{Total}−[S1]−K₂[S1] (6)

$$[ S2 ] = \frac{[ DH ]}{{K}_{1} [ S1 ]} ⇒ [ S2 ] = \frac{{C}_{S1}^{Total} - [ S1 ] - {K}_{2} [ S1 ]}{{K}_{1} [ S1 ]} \qquad ( 7 )$$

Substitution of [H2], [DH], and [S2] from Equations 3, 6 and 7 in Equation 5 leads to an expression of K₁ that can be rearranged as a quadratic equation in [S1]:

[S1]²(K₁+K₂K₁)+[S1](K₁C_{S2}+K₃+K₂K₃−K₁C_{S1}+K₂+1)−(K₃+1)C_{S1}=0 (8)

This equation is simplified by making the following substitutions:

a=(K₁+K₂K₁) (9)

b=(K₁C_{S2}^{Total}+K₃+K₂K₃−K₁C_{S1}^{Total}+K₂+1) (10)

c=(K₃+1)C_{S1}^{Total} (11)

The physical solution of the quadratic equation (i.e. positive root) is (Press, 1999):

$$\begin{matrix}[ S1 ] = \frac{- b + \sqrt{{b}^{2} - 4 ac}}{2 a} \\ or & ( 12 ) \\ [ S1 ] = \frac{2 c}{- b - \sqrt{{b}^{2} - 4 ac}} & ( 13 )\end{matrix}$$

The second equation has better numerical stability (Press, 1999).

[DH], [S2], [H1], and [H2] can then be calculated using Equations 1-3.

Determination of Net Free Energy

The net free energy of hybridization is calculated as follows:

ΔG°₃₇ₙₑₜ=−RTlnKₙₑₜ (14)

where

$${K}_{net} = \frac{[ DH ]}{[ {S1}_{single stranded} ] [ {S2}_{single stranded} ]} \qquad ( 15 )$$

where [S1 single stranded] and [S2 single stranded] are the concentrations of S1 and S2 either in the random coil state or the hairpin states, at the temperature of the simulation. Using the conservation of S1 and S2, Equation 14 is rewritten as follows:

$$Δ {G}_{T net}^{°} = - RT ln \frac{[ DH ]}{( {C}_{S1}^{Total} - [ DH ] ) ( {C}_{S2}^{Total} - [ DH ] )} \qquad ( 16 )$$

Note that ΔG°_{T net} has the unusual property that it depends on the total strand concentrations, C_{S1}^{Total} and C_{S2}^{Total}. The net free energy expresses the duplex formation equilibrium free energy corrected for secondary-structure formation in the single strands.

Determination of Net Melting Temperature

If the strands are non self-complementary two cases have to be considered depending on the relative strand concentrations:

1) If S1 is the limiting reagent (C_{S1}^{Total}<C_{S2}^{Total}), at T_{M}:

$$[ DH ] = \frac{1}{2} {C}_{S1}^{Total} \qquad ( 17 )$$

The concentrations of strands [S1] and [S2] are given by the following relations:

$$\begin{matrix}{C}_{S1}^{Total} = \frac{1}{2} {C}_{S1}^{Total} + {K}_{2} [ S1 ] + [ S1 ] & ( 18 a ) \\ [ S1 ] = \frac{{C}_{S1}^{Total}}{2 ( {K}_{2} + 1 )} & ( 18 b ) \\ {C}_{S2}^{Total} = \frac{1}{2} {C}_{S1}^{Total} + {K}_{3} [ S2 ] + [ S2 ] & ( 19 a ) \\ [ S2 ] = \frac{{C}_{S2}^{Total} - \frac{1}{2} {C}_{S1}^{Total}}{{K}_{3} + 1} & ( 19 b )\end{matrix}$$

The replacement of [S1] and [S2] in Equation 1 gives:

$$\begin{matrix}{K}_{1} = \frac{[ DH ]}{[ S1 ] [ S2 ]} = \frac{{K}_{2} + {K}_{2} {K}_{3} + {K}_{3} + 1}{{C}_{S2}^{Total} - \frac{1}{2} {C}_{S1}^{Total}} & ( 20 a ) \\ 0 = \frac{1}{2} {C}_{S1}^{Total} - {C}_{S2}^{Total} + \frac{{K}_{2} + {K}_{2} {K}_{3} + {K}_{3} + 1}{{K}_{1}} & ( 20 b )\end{matrix}$$

Using the relation ΔG°_{T}=−R T ln K, Equation 20 is arranged as follows:

$$0 = \frac{1}{2} {C}_{S1}^{Total} - {C}_{S2}^{Total} + ( {ⅇ}^{\frac{- Δ {G}_{T}^{°} ( 2 )}{RT}} + {ⅇ}^{\frac{- Δ {G}_{T}^{°} ( 2 ) - Δ {G}_{T}^{°} ( 3 )}{RT}} + {ⅇ}^{\frac{- Δ {G}_{T}^{°} ( 3 )}{RT}} + 1 ) {ⅇ}^{\frac{- Δ {G}_{T}^{°} ( 1 )}{RT}} \qquad ( 21 )$$

ΔG°_{T} can then be decomposed as ΔG°_{T}=ΔH°−TΔS° (assuming ΔC°ₚ₌₀) to obtain:

$$0 = \frac{1}{2} {C}_{S1}^{Total} + {C}_{S2}^{Total} + {ⅇ}^{\frac{- Δ {H}^{°} ( 2 ) + Δ {H}^{°} ( 1 )}{RT}} {ⅇ}^{\frac{+ Δ {S}^{°} ( 2 ) - Δ {S}^{°} ( 1 )}{R}} + {ⅇ}^{\frac{- Δ {H}^{°} ( 2 ) - Δ {H}^{°} ( 3 ) + Δ {H}^{°} ( 1 )}{RT}} {ⅇ}^{\frac{Δ {S}^{°} ( 2 ) + Δ {S}^{°} ( 3 ) - Δ {S}^{°} ( 1 )}{R}} + {ⅇ}^{\frac{- Δ {H}^{°} ( 3 ) + Δ {H}^{°} ( 1 )}{RT}} {ⅇ}^{\frac{Δ {S}^{°} ( 3 ) - Δ {S}^{°} ( 1 )}{R}} + {ⅇ}^{\frac{Δ {H}^{°} ( 1 )}{RT}} {ⅇ}^{\frac{- Δ {S}^{°} ( 1 )}{R}} \qquad ( 22 )$$

The above equation can be solved by bisection or other numerical techniques to find T. This solution is the net melting temperature.

2) If S2 is the limiting reagent (C_{S2}^{Total}<C_{S1}^{Total}), the following relation can be deduced by a similar approach:

$$0 = \frac{1}{2} {C}_{S2}^{Total} - {C}_{S1}^{Total} + \frac{{K}_{3} + {K}_{3} {K}_{2} + {K}_{2} + 1}{{K}_{1}} \qquad ( 23 )$$

Again, application of the bisection method to an equation symmetric to Equation 22 affords the net melting temperature.

If the strand S is self complementary, the reactions are described by the following equilibria:

$$\begin{matrix}\begin{matrix}S + S ⇆ DH & {K}_{1} = \frac{[ DH ]}{{[ S ]}^{2}}\end{matrix} & ( 24 ) \\ \begin{matrix}S ⇆ H & {K}_{2} = \frac{[ H ]}{[ S ]}\end{matrix} & ( 25 ) \\ {AtT}_{M} : [ DH ] = \frac{1}{4} {C}_{S}^{Total} & ( 26 )\end{matrix}$$

The strand conservation equation is:

C_{S}^{Total}=[S]+[H]+2[DH] (27)

Insertion of [H] and [DH] from Equations 25 and 26 in Equation 27 leads to:

$${C}_{S}^{Total} = 2 ( {K}_{2} + 1 ) [ S ] ⇔ [ S ] = \frac{{C}_{S}^{Total}}{2 ( {K}_{2} + 1 )} \qquad ( 28 )$$

Introduction of [S] in Equation 24 gives:

$${K}_{1} = \frac{[ DH ]}{{[ S ]}^{2}} = \frac{( {K}_{2}^{2} + 1 + 2 {K}_{2} )}{{C}_{S}^{Total}} ⇔ 0 = {K}_{2}^{2} + 2 {K}_{2} - {K}_{1} {C}_{S}^{Total} + 1 \qquad ( 29 )$$

Using the relation ΔG°_{T}=−R T ln K, Equation 29 is rearranged as follows:

$$0 = {ⅇ}^{\frac{- 2 Δ {G}_{T}^{∘} ( 2 )}{RT}} + 2 {ⅇ}^{\frac{- Δ {G}_{T}^{∘} ( 2 )}{RT}} - {C}_{S}^{Total} {ⅇ}^{\frac{Δ {G}_{T}^{∘} ( 1 )}{RT}} + 1 \qquad ( 30 )$$

ΔG°_{T} can then be decomposed as ΔG°_{T}=ΔH°−TΔS° to obtain:

$$0 = {ⅇ}^{\frac{- 2 Δ {H}^{∘} ( 2 )}{RT}} {ⅇ}^{\frac{+ 2 Δ {S}^{∘} ( 2 )}{R}} + 2 {ⅇ}^{\frac{- Δ {H}^{∘} ( 2 )}{RT}} {ⅇ}^{\frac{+ Δ {S}^{∘} ( 2 )}{R}} - {C}_{S}^{Total} {ⅇ}^{\frac{- Δ {H}^{∘} ( 1 )}{RT}} {ⅇ}^{\frac{+ Δ {S}^{∘} ( 1 )}{R}} + 1 \qquad ( 31 )$$

This equation can be solved by bisection to afford the net melting temperature.

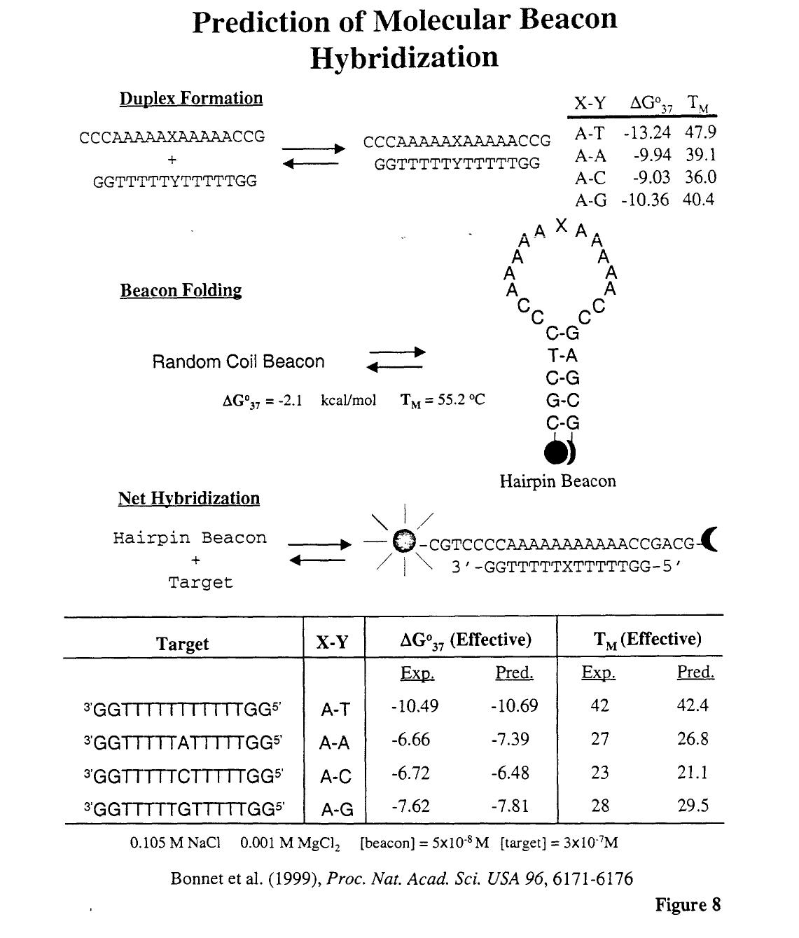

An experimentally validated example of the accuracy of the net hybridization thermodynamics is shown in FIG. 8 for molecular beacons. At the top of FIG. 8 are the predicted thermodynamics for simple duplex formation assuming no competing single strand secondary structure. Using Module 1 of the invention, these results are similar to what would be predicted using other commercial software (such as oligo 6.0), though our thermodynamic database includes the dangling end effects and salt corrections are more accurate than other software. The middle of FIG. 8 shows the single strand folding at the molecular beacon as output from DNA-MFOLD. The bottom table of FIG. 8 shows the experimentally determined Δ6 (effective) and Tm (effective) published in Bonnet et al. 1999, as well as the effective Tm and Δ6 (effective) predicted with Module 1 using the coupled equilibria calculations. Note the close agreement between experiments and predictions in the bottom table and the disagreement between experiments and the predictions using the naive simple hybridization calculation (top table of FIG. 8). Also note the good agreement in the bottom table for the fully matched A-T sequence and mismatch A-A, A-C, and A-6 sequences, thus validating the mismatch parameters.

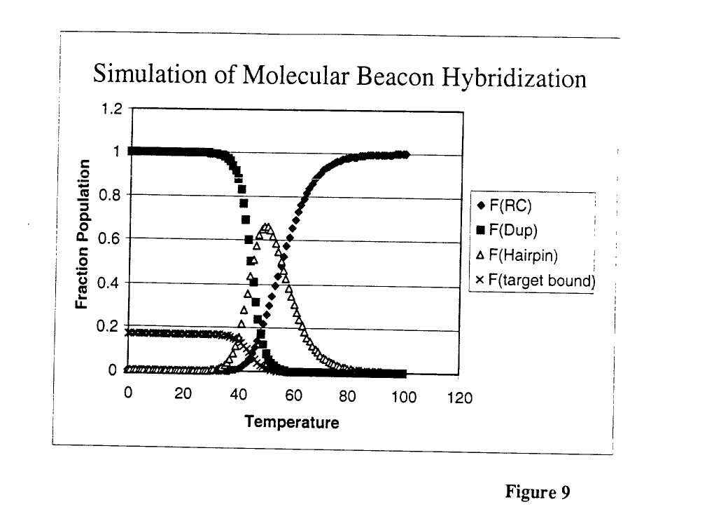

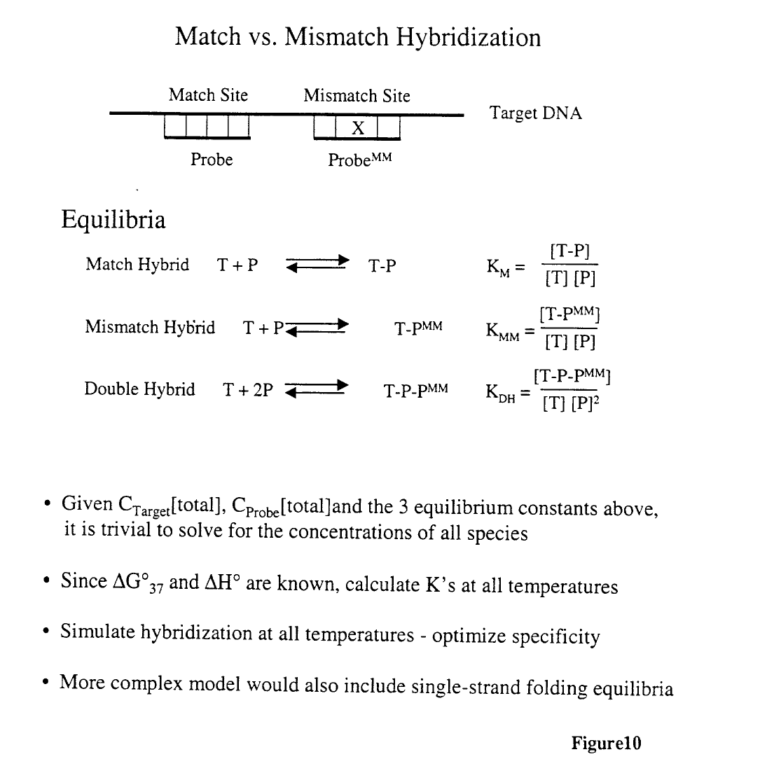

Further, the net hybridization calculations can be extended to different temperatures as shown in FIG. 9, to reveal how the concentrations of all species change with temperature. Given the extinction coefficients and fluorescence quantum yields, the concentration vs. temperature profiles shown in FIG. 9 can be used to calculate the fluorescence vs. temperature profile (not shown), thereby allowing the prediction of the temperature which produces the maximum fluorescence signal and minimum background fluorescence signal.

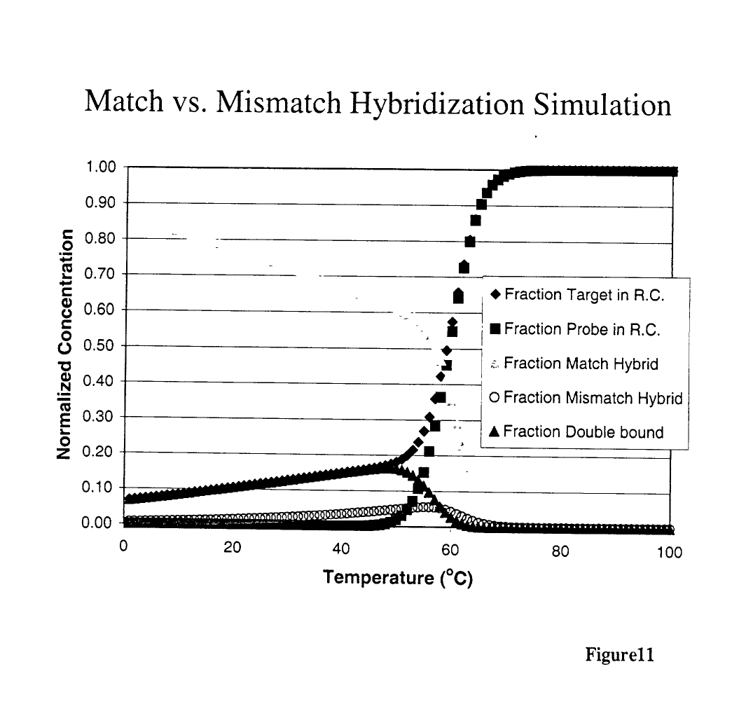

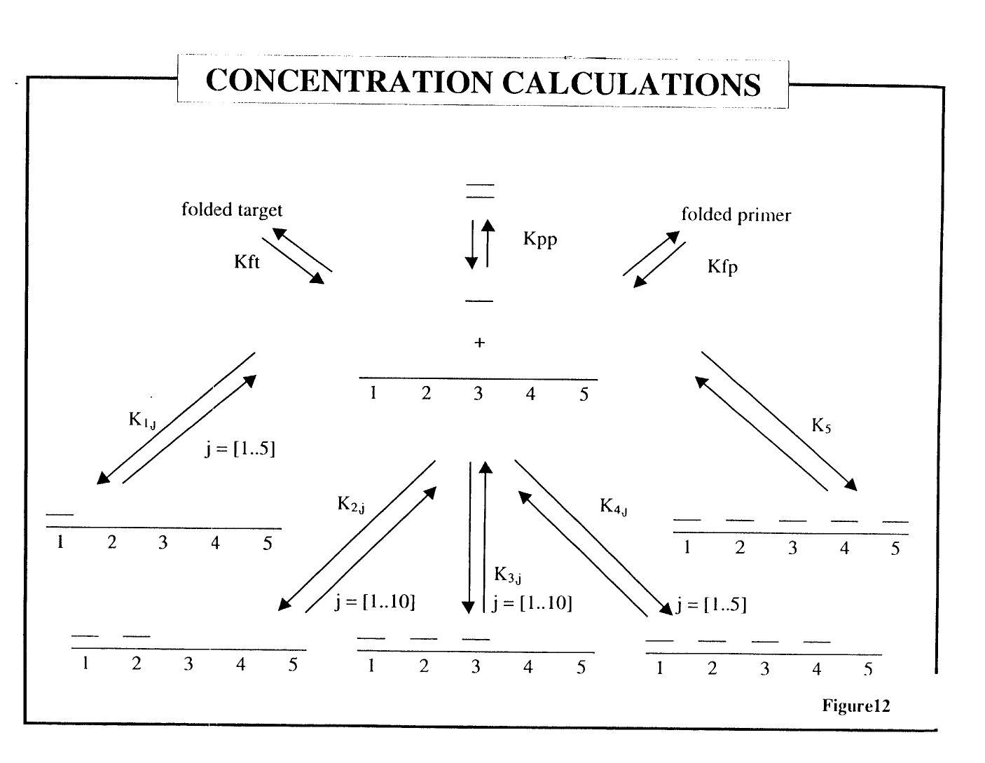

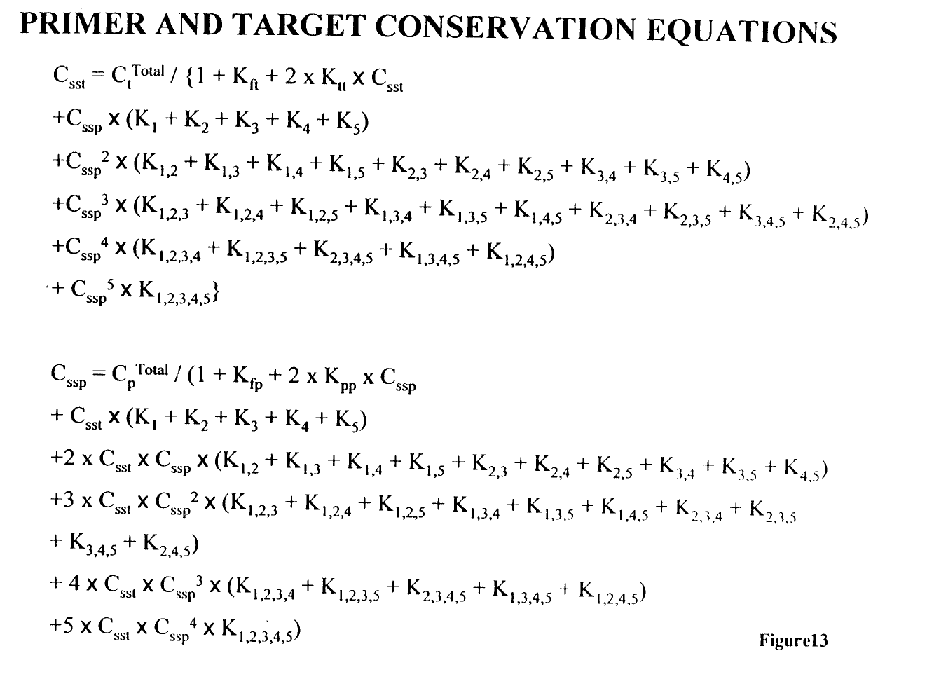

Another manifestation of the concentration calculations is for match vs. mismatch discrimination (FIG. 10), whereby the concentrations of all species at all temperatures can be calculated (FIG. 11). For the particular case shown, optimal match vs. mismatch discrimination is predicted to occur at 0° C. The concentration calculations can be generalized for cases in which molecules can form many different competing unimolecular, biomolecular, and higher order complexes (FIG. 12) using generalized equations such as shown in FIG. 13 for the five molecule case, and solved using the algorithm in FIG. 7.

Algorithm

The hybridization prediction algorithm of the present invention is based on a nearest-neighbor-model analysis of the sequences. The algorithm accounts for structural motifs including Watson-Crick base pairs (Allawi and SantaLucia, 1997; SantaLucia, 1998; Sugimoto et al., 1995; Xia et al., 1998), single internal mismatches (Allawi and SantaLucia, 1997; Allawi and SantaLucia, 1998; Allawi and SantaLucia, 1998; Allawi and SantaLucia, 1998; Kierzek et al., 1999; Peyret et al., 1999; SantaLucia, 1998), double mismatches (Allawi and SantaLucia, 1997) coaxial-stacking interfaces (disclosed herein) (Walter and Turner, 1994), terminal mismatches (disclosed herein) (Freier et al., 1986) and dangling ends (Bommarito et al., 2000; Freier et al., 1986). Once the motifs are identified and their thermodynamic contributions are added, the sum may be corrected for salt effects (sodium and magnesium) and the net hybridization is calculated when appropriate.

Algorithm Functions

A first or main module of the algorithm calculates the hybridization thermodynamics (ΔH°, ΔS°, ΔG°₃₇, T_{M}) of a given duplex. Net hybridization accounting for secondary structure in both strands is also calculated.

Parameterization

Parameters are organized in three arrays. The first array contains internal element parameters: Watson-Crick nearest neighbors and single mismatch nearest neighbors. The second array contains terminal element parameters: terminal mismatches and dangling ends. A single parameter is used to account for double mismatches except for tandem G•T mismatches, which are explicitly enumerated (Allawi & SantaLucia, 1997). The third array contains coaxial-stacking parameters (contained herein).

For DNA sequences, the thermodynamic contribution of all Watson-Crick nearest neighbors and single internal mismatches has been systematically studied (Allawi and SantaLucia, 1997; Allawi and SantaLucia, 1998; Allawi and SantaLucia, 1998; Allawi and SantaLucia, 1998; Peyret et al., 1999). A limited number of sequences containing double mismatches has also been studied (Allawi and SantaLucia, 1997). The contributions of dangling ends (Bommarito et al., 2000) have also been systematically analyzed. Salt corrections are available for sodium in the range 0.01 to 1 M (SantaLucia, 1998).

For RNA sequences, the thermodynamic contribution of all Watson-Crick nearest neighbors has been systematically studied (Xia et al., 1998). A limited number of sequences containing single mismatches has also been studied (Kierzek et al., 1999). The contribution of dangling ends and terminal mismatches has also been systematically analyzed (Freier et al., 1986). No salt correction has been developed for RNA and therefore the DNA salt corrections are assumed. These corrections are likely to be deficient in the case of RNA.

For DNA/RNA hybrids, the thermodynamic contribution of all Watson-Crick nearest neighbors has been systematically studied as well as a limited number of sequences containing single mismatches (Sugimoto et al., 1995). As no salt correction has been developed for DNA/RNA hybrids, the DNA corrections are assumed. The applicability of these corrections to DNA/RNA hybrids has not been tested. The parameter arrays are designed to easily accommodate implementation of new parameters and salt corrections including thermodynamics parameters for modified bases and denaturant effects.

Correction for Hybridization to DNA Microchips

A linear correction of the free energy is implemented in the algorithm of the invention to correct for hybridization to DNA microchips:

ΔG°₃₇(microchip)=aΔG°₃₇(solution)+b (32)

where a and b are user defined real coefficients. Fotin et al. (Fotin et al., 1998) showed that a linear relationship could be used to relate the free energies obtained for hybridization in solution and on microchip surfaces. However, the relation between thermodynamics measured in solution and thermodynamics measured using microarrays is still unclear and appears to be different depending on the manufacture and type of microarrays.

User Interface: Input and Output

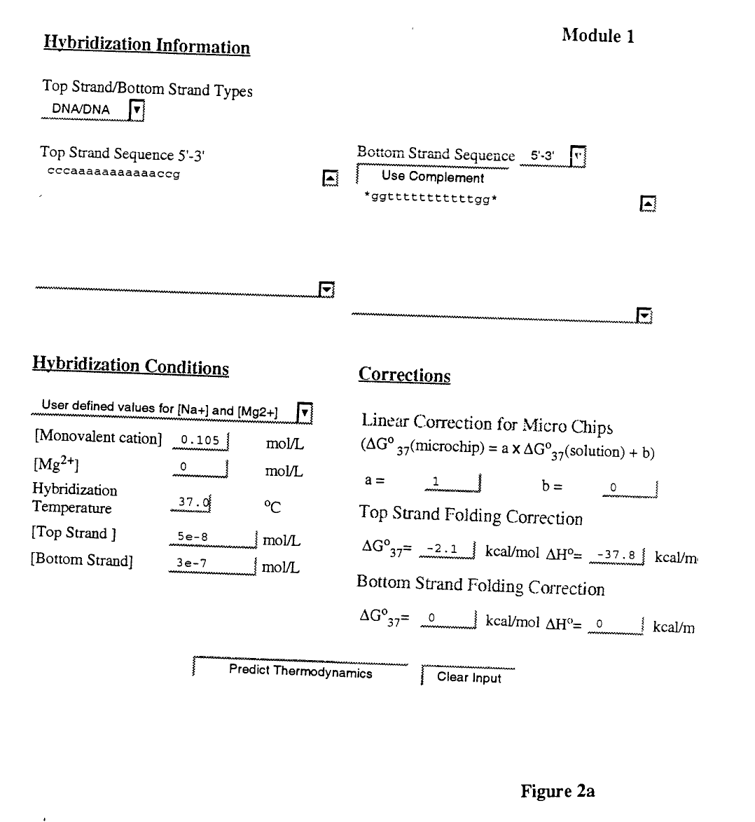

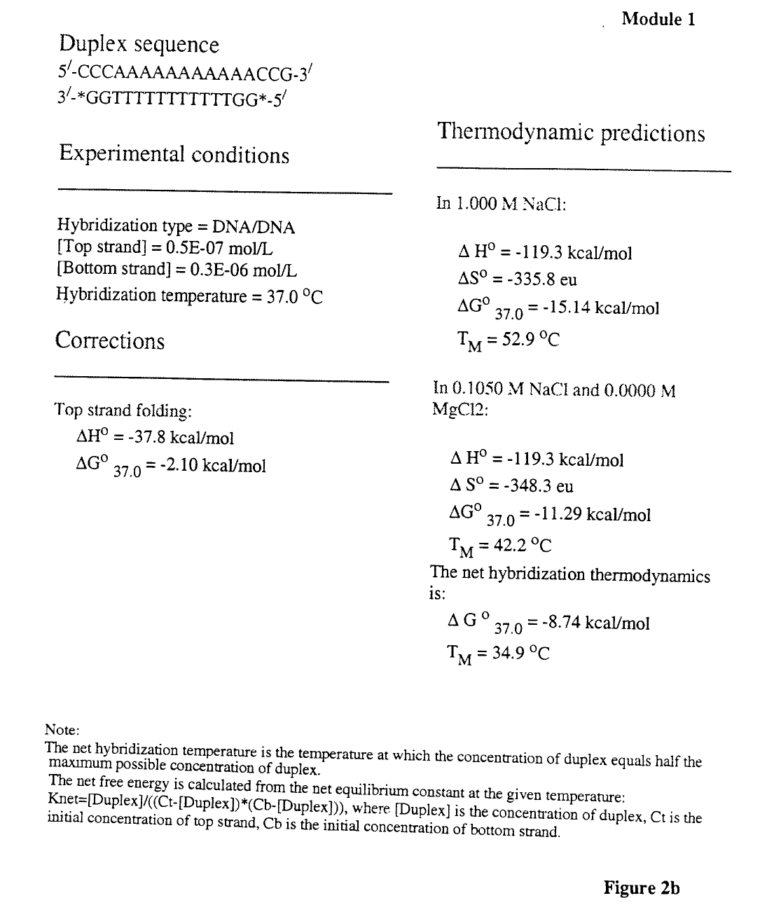

FIG. 2a shows the user interface input. The users enter the sequence of each strand, the hybridization conditions (hybridization temperature, strand concentrations, and monovalent cations and concentrations), and thermodynamic corrections for single strand folding. FIG. 2b shows the output corresponding to the input in FIG. 2a.

The algorithm can be used via the Internet at: http://jsl1.chem.wayne.edu/Hyther/hytherm1main.html. The algorithm may be written in FORTRAN 77 and run on UNIX environment or other languages and environments.

Molecular Beacons

The algorithm may be used to predict the thermodynamics of a set of literature measurements for molecular beacons (Bonnet et al., 1999). Molecular beacons are high specificity probes that are efficient for mutation analysis (Giensendorf et al., 1998) and multiplex detection of single nucleotide variations (Marras et al., 1999). The design and efficiency optimization of these beacons is helped by hybridization thermodynamics prediction. Bonnet et al. studied, the hybridization of the molecular beacon ^{5′}CGC, TCC, CAA, AAA, AAA, AAA, CCG AGC G^{3′} to a set of four different targets including a perfect match duplex, and three different duplexes containing one mismatch. Free energy and enthalpy for duplex folding may be calculated using the DNA MFOLD program (http://mfold2.wustl.edu/˜mfold/dna/form1.cgi). These parameters may then incorporated as secondary structure corrections in FIG. 2a.

The software to implement the algorithm may be written in FORTRAN, C⁺⁺, Visual Basic, HTML, and JAVA script computer languages. Two graphical user interfaces may be provided: Windows application and web browser format. The software may run on IBM/PC, Sun, and Silicon Graphics platforms.

The software may be written in several modules as described below.

A. Interactive Mode: Command Line Interface in MS-DOS

Module 1 (As Previously Described Above)

Function. Module 1 predicts the hybridization thermodynamics of a given duplex (DNA/DNA, RNA/RNA, or DNA/RNA).

Input (FIG. 2a)

Input of Sequences

1. Only the following characters are accepted: A, a, C, c, G, g, T, t, U, u, /, *, +. Single blank characters and numbers will be automatically edited, but more than one carriage return is not permitted.

2. If the duplex contains a dangling end on a strand, the sequence of the other strand should contain a * at the corresponding position. (This is very important to include for primer binding to a large target sequence). Note: The top strand must be entered in 5′ to 3′ orientation, but the bottom strand must be entered in 3′ to 5′ orientation. Also, a “+” must be added at the end of each sequence. There is a length limit of 1024 characters for sequence entries. In module 1, it is important to be sure that both sequences have the same length.

### EXAMPLE

AAAACCCCTGA+
*TTTGGGGAC*+

3. Only the bottom strand may contain coaxially stacked nucleotides. A “/” should be inserted at the site of a strand nick (i.e. between the coaxially stacked nucleotides). This feature is useful for predicting stacked hybridization stability.

### EXAMPLE

AAAACCCCC+
TTTT/GGGG+

Input of Salt and Strand Concentrations

The monovalent salt should be the sum of all monovalent cation concentrations in a solution in units of molarity. For example, a solution of 100 mM KCl, 50 mM NaCl, 10 mM Na₂PO₄, 0.1 mM Na₂EDTA would account for a total of 0.1702 M monovalent. The thermodynamic predictions are applicable over a salt range of 0.01 to 1 M monovalent cation. The correction applied is from SantaLucia (1998) Proc. Natl. Acad. Sci. 95, 1460. The sodium correction applies for oligonucleotides with fewer than about 30 base pairs. For longer duplexes a polymer correction is required, but this is not currently implemented.

Strand concentrations are entered in units of molarity. The program will accept virtually any physically relevant strand concentration.

Hybridization temperature is in Celsius degrees. The limits are 0 to 100 degrees.

Special corrections for single-stranded secondary structure and for surface corrections for hybridization arrays can be input. The units for input ΔG° are kcal/mol. To determine estimates of single-strand folding energies, see Michael Zuker's RNA or DNA-MFOLD servers (see http://mfold2.wsutl.edu/˜mfold/dna/form1.cgi). The current thermodynamic prediction software incorporates the special corrections for single-stranded secondary structure and for surface corrections for hybridization arrays.

For DNA chip arrays, a linear correction can be applied. The user inputs the slope and intercept coefficients. Based on the work of Mirzabekov group, a slope of +1.1 and intercept of +3.2 are appropriate (see Fotin et al. (1998) Nucleic Acids Res. 26, 1515-1521).

Output (FIG. 2b)

Module 1 outputs the hybridization thermodynamics at 1.0 M NaCl and 37° C. (the conditions under which the thermodynamic predictions are most accurate), under the salt temperature conditions specified by the user, and also displays the net hybridization Tm and ΔG° if the user specifies that special corrections are needed (this allows for single-strand secondary structure of both the target and probe DNA to be accounted and for surface effects of chip arrays). Predictions of ΔG°, ΔH°, ΔS°, and Tm are provided.

Module 2

Function. Module 2 finds the best primers of given length complementary to a long target nucleic acid. DNA/DNA, RNA/RNA, DNA/RNA hybridization types are accepted. The user selects the number of primers to output, and the program finds the most stable primers and gives their hybridization position and thermodynamics of each primer.

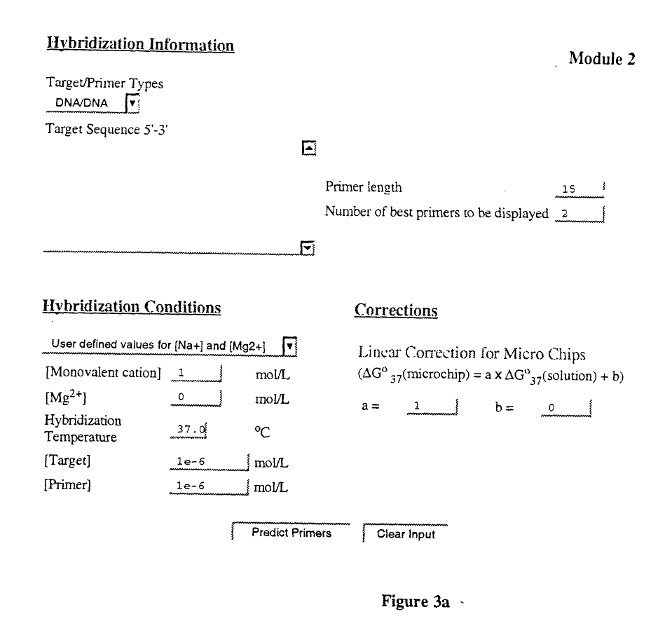

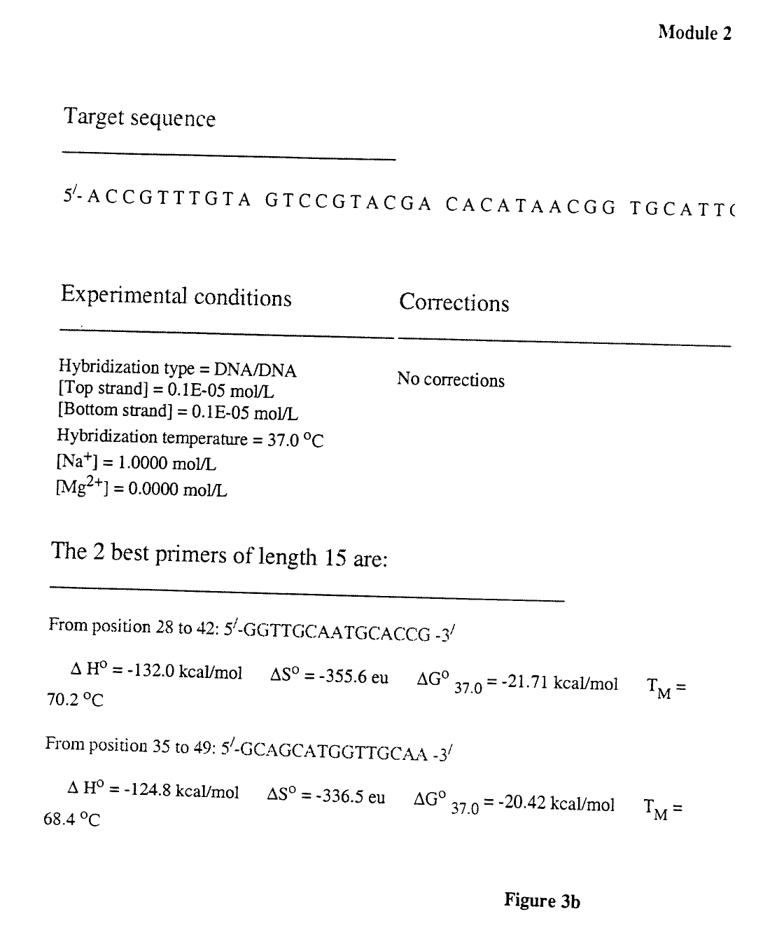

Input (FIG. 3a)

Input of Salt and Strand Concentrations

The input of strand and salt concentrations is similar to Module 1.

Input of Sequences

The target sequence is input as in Module 1.

Output (FIG. 3b)

Primer Length and Number of Best Primers

Module 2 displays “number of best primers” best primers of length “primer length” in order of decreasing stability.

Output

Module 2 outputs “number of best primers” best primers of length “primer length” in order of decreasing stability along with their hybridization thermodynamics.

Module 3

Function. Module 3 walks a given primer along a given target and finds the thermodynamics for the best target/primer complex and for the competitive target/primer complexes: DNA/DNA, RNA/RNA, DNA/RNA, hybridization types are accepted.

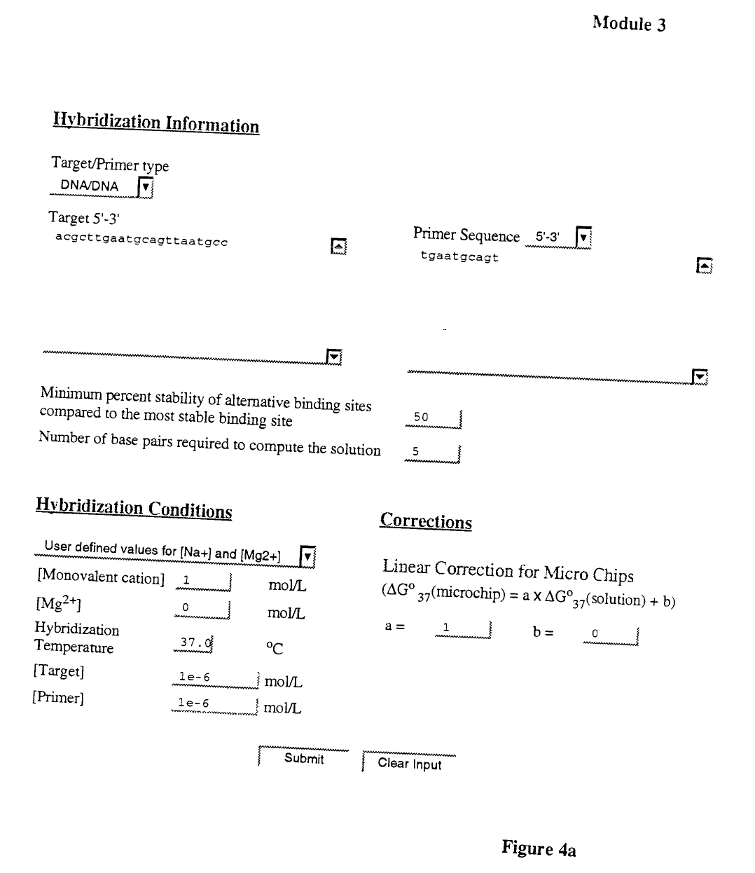

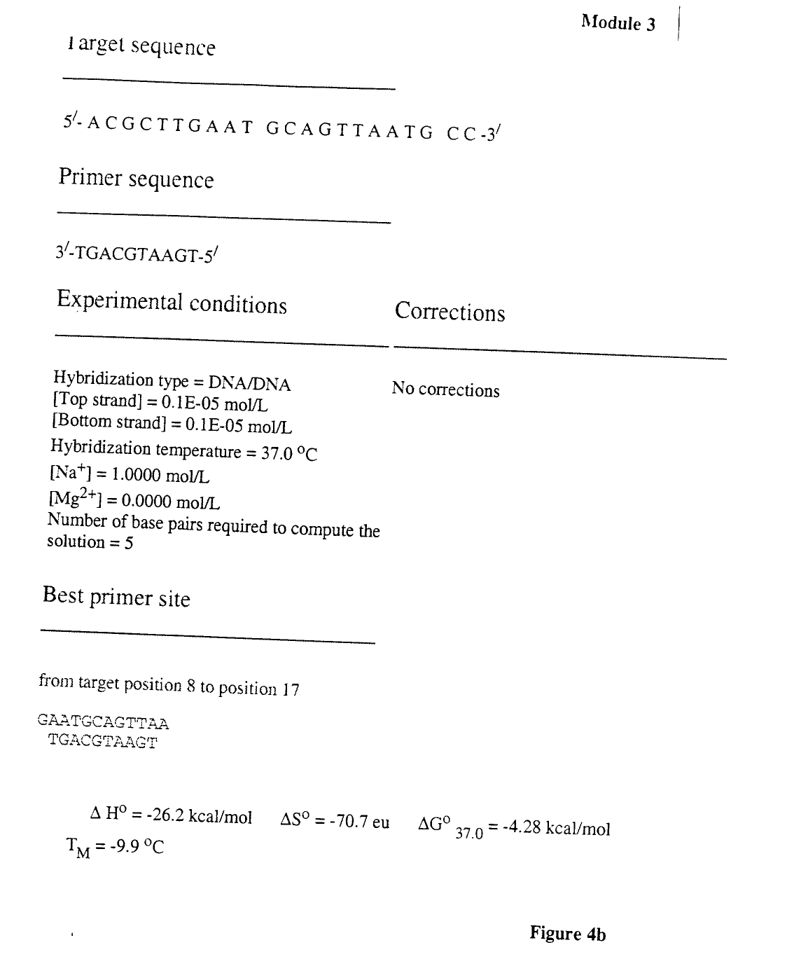

Input (FIG. 4a)

Input of Sequences

The input is similar to Module 1. The target has to be longer than the primer.

Input of Salt and Strand Concentrations

The input of salt and strand concentrations is similar to Module 1.

Percent Stability p of Alternative Binding Sites Compared to the Most Stable Binding Site

This parameter excludes all competitive sites that are not within the defined percent of the best primer stability. If the best primer stability is −5 kcal/mol and p=10 then any competitive site of energy higher than −5+(10/100*5)=−4.5 kcal/mol will not be displayed.

Number of Base Pairs Required to Compute the Solution

This parameter excludes all competitive sites that contain less Watson-Crick base pairs than the defined value.

Output (FIG. 4b)

Module 3 outputs the best primer binding site and the competitive binding sites that pass the filtering criteria (percent stability p of alternative binding sites compared to the most stable binding site and number of best primers).

Module 4

Function. Batch mode calculations (see below).

Module 5

Function. Module 5 is a combination of Modules 2 and 3 and finds the n best primers of given length complementary to a given section of a target and display the thermodynamics of the target/primer system(s). Then, each best primer is walked along the whole target to find the competitive hybridization sites. The thermodynamics of the target/primer systems at these alternative sites is then displayed. DNA/DNA, RNA/RNA, DNA/RNA, hybridization types are accepted.

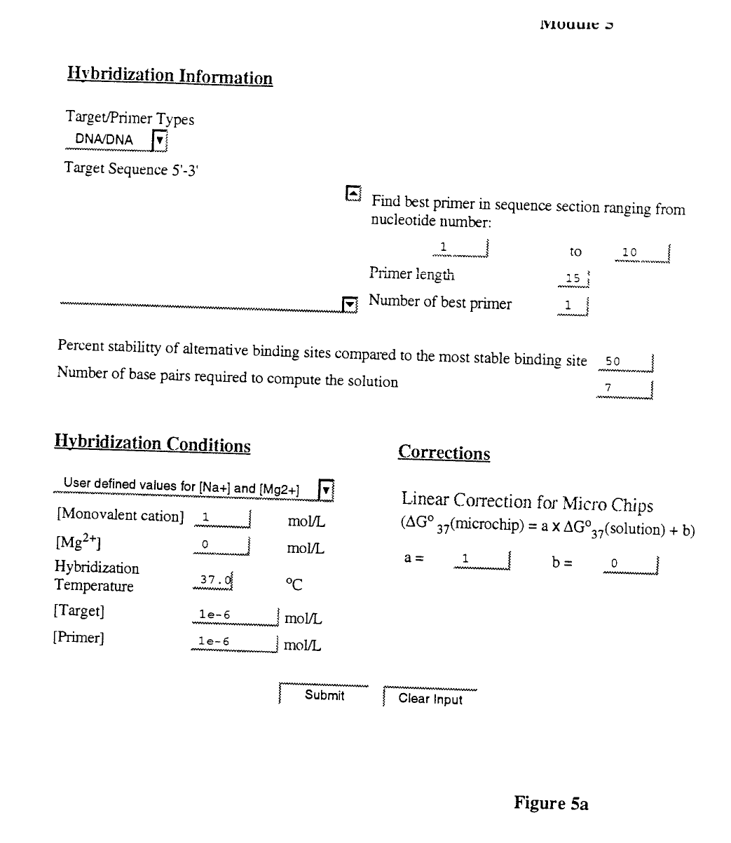

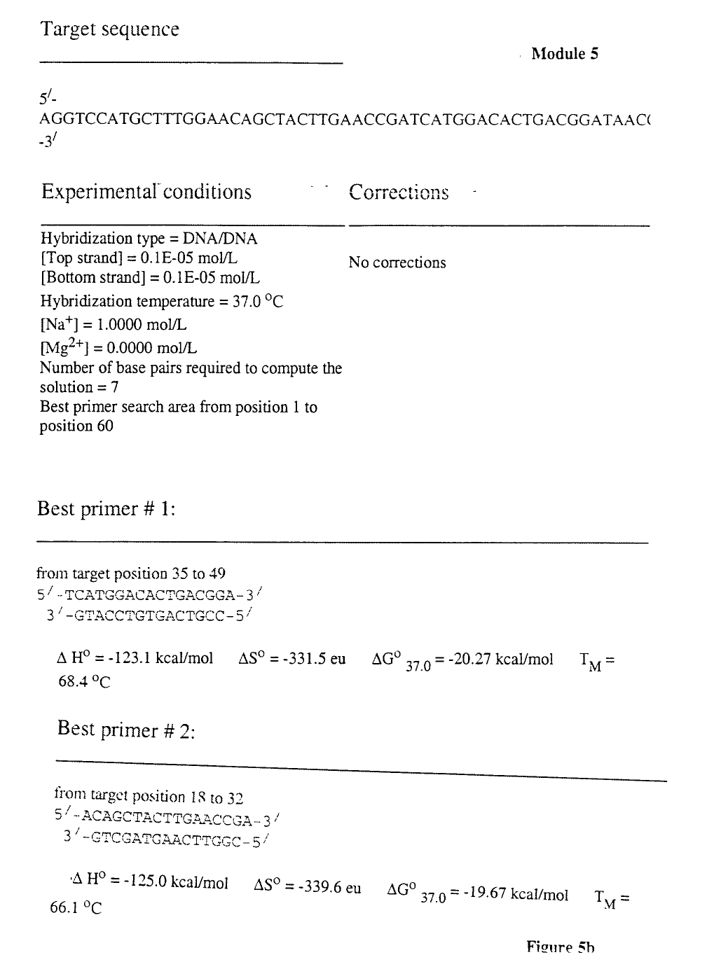

Input (FIG. 5a)

Input of Sequences

The target sequence is input as in Module 1.

Input of Salt and Strand Concentrations

The input of salt and strand concentrations is similar to Module 1.

Sequence Section Where to Find the Best Primers

Module 5 finds the best primers in the target region ranking from “position of initial nucleotide” to “position of final nucleotide”. Note that Module 5 then looks for competitive sites of each best primers in the whole target.

Percent Stability of Alternative Binding Sites Compared to the Most Stable Binding Site

The function of this parameter is the same as in Module 3. This parameter is input for each best primer corresponding to the “number of best primer” specified.

Number of Base Pairs Required to Compute the Solution

The function of this parameter is the same as in Module 3. This parameter is input for each best primer corresponding to the “number of best primer” specified.

Output (FIG. 5b)

Primer Length and Number of Best Primers

Module 5 displays “number of best primers” best primers of length “primer length” by order of decreasing stability.

Output

Module 5 displays “number of best primers” best primers and their competitive sites by order of stability along with their hybridization thermodynamics. The best primer and its ranked competitive hybridization sites are listed first. Then, the second best primer is listed with its competitive hybridization sites.

Module 6

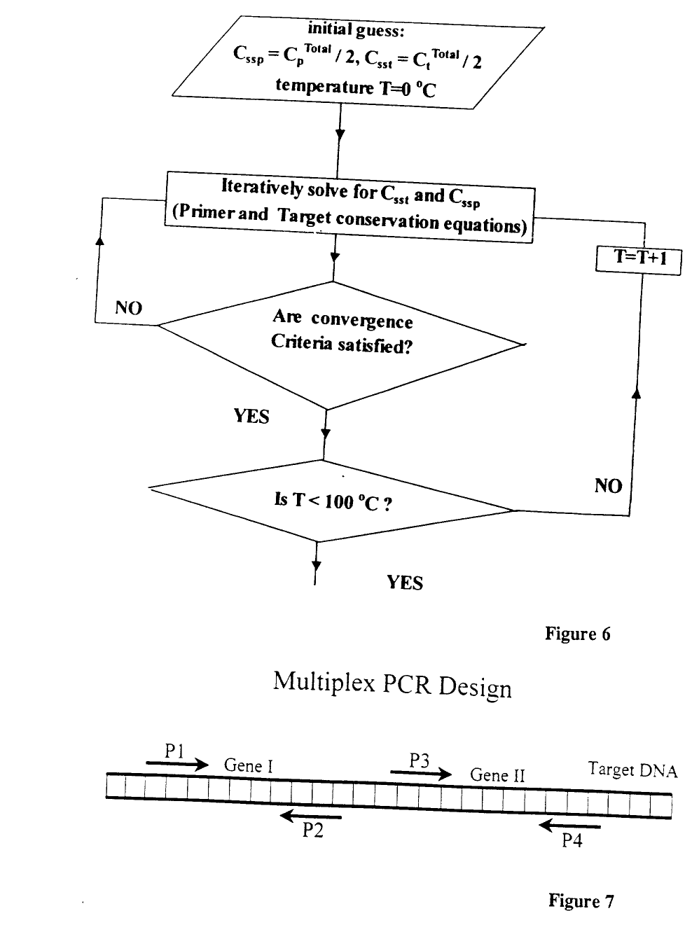

Function. Module 6 is similar to Module 3 and walks a given primer along a given target and finds the thermodynamics for the best target/primer complex and for the competitive target/primer complexes: DNA/DNA, RNA/RNA, DNA/RNA, hybridization types are accepted. Then, Module 6 simulates the concentration of every species at every degree from 1 to 100° C., as illustrated in FIG. 6.

Input (Not Shown)

Input of Sequences

The input is similar to Module 1. The target has to be longer than the primer.

Input of Salt and Strand Concentrations

The input of salt and strand concentrations is similar to Module 1.

Percent Stability p of Alternative Binding Sites

Compared to the Most Stable Binding Site

This parameter excludes all competitive sites that are not within the defined percent of the best primer stability. If the best primer stability is −5 kcal/mol and p=10, then any competitive site of energy higher than: −5+(10/100*5)=−4.5 kcal/mol will not be displayed.

Number of Base Pairs Required to Compute the Solution

This parameter excludes all competitive sites that contain less Watson-Crick base pairs than the defined value.

Correction for Target/Target Interaction, Target Folding, Primer/Primer Interaction and Primer Folding

The user is asked if he wants to correct for the interactions above. If the answer is “y”, the user is prompted for ΔH°₃₇ corresponding to the interaction. Secondary structure thermodynamics can be determined using the Zuker algorithm as discussed in Module 1 section.

Output (Not Shown)

Concentration Output Filename

The results from the concentration simulations (concentration of species at every temperature) are saved in this file.

Output

Module 6 outputs the best primer binding site and the competitive binding sites that pass the filtering criteria (percent stability p of alternative binding sites compared to the most stable binding site and number of best primers). The concentration simulations are saved in a file specified by the user.

Module 7

Function. Module 7 is a combination of Modules 2 and 5 and finds the n best primers of given length complementary to a given section of a target and display the thermodynamics of the target/primer system(s). Then, each best primer is walked along the whole target to find the competitive hybridization sites. The thermodynamics of the target/primer systems at these alternative sites is then displayed. DNA/DNA, RNA/RNA, DNA/RNA hybridization types are accepted. Then, Module 7, like Module 6, simulates the concentration of every species at every degree from 1 to 100° C., as illustrated in FIG. 6.

Input (Not Shown)

Input of Sequences

The target sequence is input as in Module 1.

Input of Salt and Strand Concentrations

The input of salt and strand concentrations is similar to Module 1.

Sequence Section where to Find the Best Primers

Module 7 finds best primers in the target region ranking from “position of initial nucleotide” to “position of final nucleotide.” Note that Module 7 then looks for competitive sites of each best primers in the whole target.

Percent Stability of Alternative Binding Sites Compared to the Most Stable Binding Site

The function of this parameter is the same as in Module 3. This parameter is input for each best primer corresponding to the “number of best primer” specified.

Number of Base Pairs Required to Compute the Solution

The function of this parameter is the same as in Module 3. This parameter is input for each best primer corresponding to the “number of best primer” specified.

Correction for Target/Target Interaction, Target Folding, Primer/Primer Interaction and Primer Folding

For each best primer, the user is asked if he wants to correct for the interactions above. If the answer is “y”, the user is prompted for ΔH° and ΔG°₃₇ corresponding to the interaction. Secondary structure thermodynamics can be determined using the Zuker algorithm as discussed in Module 1 section.

Concentration Output Filenames

For each best primer, the results from the concentration simulations (concentration of species at every temperature) are saved in this file. The user has to select a different filename for each best primer.

Output (Not Shown)

Primer Length and Number of Best Primers

Module 7 displays “number of best primers” best primers of length “primer length” by order of decreasing stability.

Module 7 displays “number of best primers” best primers and their competitive sites by order of stability along with their hybridization thermodynamics. The best primer and its ranked competitive hybridization sites are listed first. Then, the second best primer is listed with its competitive hybridization sites. For each best primer, a file named by the user contains the concentration simulations.

Module 7 allows the user to design optimal primers for applications where multiple simultaneous hybridization reactions are occurring, including match vs. mismatch hybridization, molecular beacons, DNA oligonucleotide arrays, and multiplex PCR.

One commercially important example for the use of Module 7 for primer design in a complex hybridization solution is Multiplex PCR, as shown in FIG. 7. Module 7 allows the user to design optimal primers for Multiplex PCR where multiple primers have equal stabilities in binding to the target DNA. Several primers must be designed to specifically bind to different sites on target DNA at a given temperature with minimal background binding to mismatch sites and with minimal cross-hybridization between pairs of primers.

Module 7 minimiizes potential primer dimer formation and mismatch hybridization for all combinations of input primers. Module 7 optimizes primer sequence position, length, and concentration for each primer in relation to all other species in solution and provides a hybridization profile at all temperatures from 0 to 100° C.

Batch Mode

Module 4

Function. Module 4 allows any of the previous modules to be run in batch mode using text files to submit the input and having the data output as text files also.

Type of Input Files

There are two types of input files: 1) parameter input file, and 2) sequence input file. Parameter input files describe what modules to run with what hybridization parameters and on how many sequences to run them. Example of parameter input files for each module with comments are given in the “Batch mode parameter files folder.” Sequence files contain the sequences that are going to be hybridized in the conditions described by the parameter input files. Examples of parameter input files for each module with comments are given in the “Batch mode sequence files folder.”

Note that a parameter file can successively run different modules on various different sequences.

The user is successively asked for the names of the parameter input file, the sequence input file and the thermodynamic data output file. Note that these files have to be in the directory containing the executable version of the software. Output files will also be created in this same directory. Names of the concentration simulation files are specified in the parameter input files.

Examples of Batch Mode Parameter Files

Comments in parentheses describe the meaning of each entry (note that an actual parameter file must not contain these comments).

| | |
|---|---|
| DUP | (Module 1: Simple duplex calculations) |
| 1 | (Number of sequences to apply this parameter file to) |
| 1 | (Monovalent cations concentration mol/L)) |
| 1 | Mg²⁺ concentration mol/L) |
| 37.0 | (Hybridization temperature) |
| 4e-4 | (Top strand concentration mol/L) |
| 4e-4 | (Bottom strand concentration mol/L) |
| 1 | (Correction for microchips: slope) |
| 0 | (Correction for microchips: intercept) |
| 0 | (Correction for top strand folding: ΔG°₃₇) |
| 0 | (Correction for top strand folding: ΔH°₃₇) |
| 0 | (Correction for bottom strand folding: ΔG°₃₇) |
| 0 | (Correction for bottom strand folding: ΔH°₃₇) |
| END | (End of file required) |
| NBP | (Module 2: N-best primers) |
| 1 | (Number of sequences to apply this parameter file to) |
| 1 | (Monovalent cations concentration mol/L) |
| 1 | (Mg²⁺ concentration mol/L) |
| 37 | (Hybridization temperature) |
| 4e-4 | (Top strand concentration mol/L) |
| 4e-4 | (Bottom strand concentration mol/L) |
| 4 | (Primer Length) |
| 3 | (Number of best primers) |
| 1 | (Correction for microchips: slope) |
| 0 | (Correction for microchips: intercept) |
| END | (End of file required) |
| PWA | (Module 3 primer walk match vs. mismatch sites identification) |
| 1 | (Number of sequences to apply this parameter file to) |
| 1 | (Monovalent cations concentration mol/L) |
| 1 | (Mg²⁺ concentration mol/L) |
| 37 | (Hybridization temperature) |
| 4e-4 | (Top strand concentration mol/L) |
| 4e-4 | (Bottom strand concentration mol/L) |
| 90 | (Percent window of best primer stability for alternative sites) |
| 2 | (Number of WC base pairs required to compute the solution) |
| cgcg+ | (Primer sequence, + required) |
| 1 | (Correction for microchips: slope) |
| 0 | (Correction for microchips: intercept) |
| END | (End of file required) |
| BPW | (Module 5 displays “number of best primers” best primers and their |
| | competitive sites by order of stability along with their hybridization |
| | thermodynamics) |
| 1 | (Number of sequences to apply this parameter file to) |
| 1 | (Lower limit of primer search area) |
| 10 | (Upper limit of primer search area) |
| 1 | Mg²⁺ concentration mol/L) |
| 37 | (Hybridization temperature) |
| 4e-4 | (Top strand concentration mol/L) |
| 4e-4 | (Bottom strand concentration mol/L) |
| 4 | (Primer length) |
| 1 | (Number of best primers) |
| 1 | (Correction for microchips: slope) |
| 0 | (Correction for microchips: intercept) |
| 800 | (Percent window of best primer stability for alternative sites) |
| 2 | (Number of WC base pairs required to compute the solution) |
| END | (End of file required) |
| PWC | (Module 6 primer walk with concentration calculations) |
| 1 | (Number of sequences to apply this parameter file to) |
| 1 | (Monovalent cations concentration mol/L) |
| 1 | Mg²⁺concentration mol/L) |
| 37 | (Hybridization temperature) |
| 4e-4 | (Top strand concentration mol/L) |
| 4e-4 | (Bottom strand concentration mol/L) |
| 90 | (Percent window of best primer stability for alternative sites) |
| 2 | (Number of WC base pairs required to compute the solution) |
| cgcg+ | (Primer sequence, + required) |
| 1 | (Correction for microchips: slope) |
| 0 | (Correction for microchips: intercept) |
| 0 | (Correction for target folding: ΔG°₃₇) |
| 0 | (Correction for target folding: ΔH°) |
| 0 | (Correction for target/target interaction: ΔG°₃₇) |
| 0 | (Correction for target/target interaction: ΔH° |
| 0 | (Correction for primer folding: ΔG°₃₇) |
| 0 | (Correction for primer folding: ΔH°) |
| 0 | (Correction for primer/primer interaction: ΔG°₃₇) |
| 0 | (Correction for primer/primer interaction: ΔH°) |
| outconc | (Concentration output file name) |
| END | (End of file required) |
| BWC | (Module 7, N-best primers, primer walk, and concentration |
| | calculations) |
| 1 | (Number of sequences to apply this parameter file to) |
| 1 | (Lower limit of primer search area) |
| 10 | (Upper limit of primer search area) |
| 1 | (Monovalent cations concentration mol/L) |
| 0 | Mg²⁺ concentration mol/L) |
| 37 | (Hybridization temperature) |
| 4e-4 | (Top strand concentration mol/L) |
| 4e-4 | (Bottom strand concentration mol/L) |
| 4 | (Primer length) |
| 1 | (Number of best primers) |
| 1 | (Correction for microchips: slope) |
| 0 | (Correction for microchips: intercept) |
| 800 | (Percent window of best primer stability for alternative sites) |
| 2 | (Number of WC base pairs required to compute the solution) |
| 0 | (Correction for target folding: ΔG°₃₇) |
| 0 | (Correction for target folding: ΔH°) |
| 0 | (Correction for target/target interaction: ΔG°₃₇) |
| 0 | (Correction for target/target interaction: ΔH°) |
| 0 | (Correction for primer folding: ΔG°₃₇) |
| 0 | (Correction for primer folding: ΔH°) |
| 0 | (Correction for primer/primer interaction: ΔG°₃₇) |
| 0 | (Correction for primer/primer interaction: ΔH°) |
| outconc | (Concentration output file name) |
| END | (End of file required) |
| PPW | (Module 8: walk a primer along itself to find interaction sites. PWA |
| | with probe = primer) |
| 1 | (Number of sequences to apply this parameter file to) |
| 1 | (Monovalent cations concentration mol/L) |
| 1 | Mg²⁺ concentration mol/L) |
| 37 | (Hybridization temperature) |
| 4e-4 | (Primer concentration mol/L) |
| 900 | (Percent window of best primer stability for alternative sites) |
| 2 | (Number of WC base pairs required to compute the solution) |
| END | (End of file required) |

Examples of Batch Mode Sequence Files

For Module 1: Dup

| | |
|---|---|
| 1 | (Sequence number) |
| agcgca+ | (Top strand sequence) |
| tcgcgt+ | (Bottom strand sequence) |

For Module 2: NBP

| | |
|---|---|
| 1 | (Sequence number) |
| agcgca+ | (Target sequence) |

For Module 3:

| | |
|---|---|
| 1 | (Sequence number) |
| cgcctgcggccc+ | (Target sequence) |

For Module 5: bpw

| | |
|---|---|
| 1 | (Sequence number) |
| cgcctgcgccc+ | (Target sequence) |

For Module 6: pwc

| | |
|---|---|
| 1 | (Sequence number) |
| agcgca+ | (Target sequence) |

For Module 7: bwc

| | |
|---|---|
| 1 | (Sequence number) |
| agcgca+ | (Target sequence) |

For Module 8: ppw

| | |
|---|---|
| 1 | (Sequence number) |
| agcgca+ | (Primer sequence) |

Example of Batch Mode Parameter and Sequence Files to Run Different Modules Successively

Parameter File:

| | |
|---|---|
| DUP | (Executes Module 1) |
| 2 | (Apply to Module 1 to 2 sequence sets) |
| 0.05 | |
| 1.5e−3 | |
| 37.0 | |
| 1e−6 | |
| 2e−7 | |
| 1 | |
| 0. | |
| −2.12 | |
| −37.3 | |
| 0 | |
| 0 | |
| PWC | (Executes Module 6) |
| 1 | (Apply to Module 6 to 1 sequence set) |
| 0.16 | |
| 0.0025 | |
| 37 | |
| 10e−9 | |
| 1e−9 | |
| 800 | |
| 8 | |
| TCGAACGTAC+ | |
| 1 | |
| 0 | |
| 0 | |
| 0 | |
| 0 | |
| 0 | |
| 0 | |
| 0 | |
| 0 | |
| 0 | |
| outwash | |
| DUP | (Executes Module 1) |
| 4 | (Apply to Module 1 to 4 sequence sets) |
| 1 | |
| 0 | |
| 37.0 | |
| 1e−6 | |
| 1e−6 | |
| 1 | |
| 0 | |
| 0 | |
| 0 | |
| 0 | |
| 0 | |
| END | |

Other modules can be Similarly Appended.

| | |
|---|---|
| Sequence File | |
| 1 | (input for Module 1) |
| ttgcctaggggaccaggtccaact+ | |
| aacggatcccctggtccaggttga+ | |
| 2 | (input for Module 1) |
| ttgcctaggggaccaggtccaact+ | |
| aacggatcccctggtccaggttga+ | |
| 3 | (input for Module 6) |
| CAGCTTGCATGAAAAGCTTGCGTGT+ | |
| 4 | (input for Module 1) |
| AAAAAA+ | |
| TTTTTT+ | |
| 5 | (input for Module 1) |
| acgcgc+ | |
| tgcgcg+ | |
| 6 | (input for Module 1) |
| gggaaagggg+ | |
| *cctttccc*+ | |
| 7 | (input for Module 1) |
| tttaaattt+ | |
| aaatttaaa+ | |
| 8 | (input for Module 1) |
| cgcgtgagggcc+ | |
| gcgctctccccgg+ | |

Parameterization of the Algorithm of the Invention

Caution: RNA/RNA and DNA/RNA duplexes contain motifs for which no literature data are available. In these cases, DNA/DNA parameters are assumed. Therefore, predictions might be inaccurate. Users are encouraged to use this program with caution and discernment.

No data are available for the following motifs:

| | |
|---|---|
| RNA/RNA | single mismatches |
| RNA/DNA | single mismatches |
| | dangling ends |
| | terminal mismatches |
| | Single mismatches |

Double mismatch parameters are estimated for all types of duplexes (DNA/DNA, RNA/RNA, DNA/RNA).

DNA THERMODYNAMIC PARAMETERS

| Watson-Crick nearest-neighbors | | |
|---|---|---|
| SantaLucia, Allawi, and Seneviratne | 12 parameters | 108 sequences |
| (1996) Biochemistry 35, 3555; | | |
| Allawi and SantaLucia (1997) | | |
| Biochemistry 36, 10581 | | |
| Single mismatch nearest-neighbors | | |
| Allawi and SantaLucia (1997) | 44 parameters | 180 sequences |
| Biochemistry 36, 100581; | | |
| Allawi and SantaLucia (1997) | Allawi and SantaLucia (1998) | |
| | Biochemistry 37, 2170 | |
| Nucleic Acids Res., 26, 2694; Peyret | Allawi and SantaLucia (1998) | |
| | Biochemistry 37, 9435 | |
| et al., (1999) Biochemistry 38, 3468 | | |
| Terminal Mismatch nearest- | | |
| neighbors | | |
| (Appendix) | 48 parameters | 48 sequences |
| Dangling end nearest-neighbors | | |
| S. Bommarito, Peyret, SantaLucia | 16 parameters | 16 sequences |
| (2000) Nucleic Acids Res. 28, 1929- | | |
| 1934. | | |
| Na⁺ dependence law | | |
| SantaLucia (1998) Proc. Natl. Acad. | 1 parameter | 86 sequences |
| Sci. USA 95, 1460-1465 | | |

RNA THERMODYNAMIC PARAMETERS

| | |
|---|---|
| Watson-Crick nearest-neighbors | |
| Xia et al. (1998) Biochemistry 37, 14719 | 12 parameters |
| Single Mismatches | |
| Kierzek, Burkard, and Turner (1999) | 44 parameters |
| Biochemistry 38, 14214-14223 | |
| Terminal mismatch nearest-neighbors | |
| Freier et al. (1986) Proc. Natl. Acad. Sci. USA 83, | 48 parameters |
| 9373 | |
| Dangling end nearest-neighbors | |
| Freier et al. (1986) Proc. Natl. Acad. Sci. USA 83, | 32 parameters |
| 9373 | |
| Coaxial stacking nearest-neighbors | |
| Walter and Turner (1994) Biochemistry 33, 12715 | 16 parameters |
| Loop parameters | |
| Matthews et al. (1999) J. Mol. Biol. 288, 911-940 | |

HYBRID DNA/RNA THERMODYNAMIC PARAMETERS

| | |
|---|---|
| Watson-Crick nearest-neighbors | |
| Sugimoto et al. (1995) Biochemistry 34, 11211 | 17 parameters |
| rU · dG and rG · dT mismatches | |
| Sugimoto et al. (1997) Nucleic Acids Symp. Ser. 37, 199 | |

DNA LOOP THERMODYNAMIC PARAMETERS

Hairpins
Hilbers et al. (1985) Biochie 67, 685-695
Blommers et al. (1989) Biochemistry 28, 7491-7498
Antao et al. (1991) Nucleic Acids Res. 19, 5901-5905
Antao et al. (1991) Nucleic Acids Res. 20, 819-824
Senior et al. (1988) Proc. Natl. Acad. Sci. USA 85, 6242-6246
Bulges
LeBlanc and Morden (1991) Biochemistry 30, 4042-4047
Zieba et al. (1991) Biochemistry 30, 8018-8026
Ke et al. (1995) Biochemistry 34, 4593-4600
Turner, D.H. (1992) Curr. Opin. Struc. Biol. 2, 334-337
Multibranched Loops
Kadrmas et al. (1995) Nucleic Acids Res. 23, 2122
Lilley and Hallam (1984) J. Mol. Biol. 180, 179-200
Lu et al. (1991) J. Mol. Biol. 223, 781-789
Ladbury et al. (1994) Biochemistry 33, 6828-6833
Leontis et al. (1991) Nucleic Acids Res. 19, 759-766
The parameters for multibranched loops are from a best fit analysis of
secondary structure predictions vs. experiments as done by Jaeger et al. for
RNA (Jaeger et al. (1989) PNAS 86, 7706-7710). The current parameters
for multibranched loops neglect the sequence and complicated length
dependence described by Leontis and coworkers, but approximate 4-way
junctions fairly well. Implementation of more complicated rules will
require modification of the MFOLD algorithm.

While the best mode for carrying out the invention has been described in detail, those familiar with the art to which this invention relates will recognize various alternative designs and embodiments for practicing the invention as defmed by the following claims.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

What is claimed is:

1. A method for predicting nucleic acid hybridization thermodynamics, the method comprising:
providing a database of thermodynamics parameters;
receiving hybridization information which represents at least one sequence;
receiving correction data;
receiving a first set of data which represents hybridization conditions; and
calculating hybridization thermodynamics including net hybridization thermodynamics based on the hybridization information, the thermodynamic parameters, the correction data and the first set of data.

2. The method as claimed in claim 1 wherein the hybridization thermodynamics of individual single stranded, bimolecular and higher order complexes are statistically weighted in a numerical process and the equilibrium concentration of each species is output.

3. The method as claimed in claim 2 wherein the correction data includes folding correction data.

4. The method as claimed in claim 2 wherein the correction data includes linear correction data.

5. The method as claimed in claim 1 wherein the thermodynamic parameters include DNA thermodynamic parameters.

6. The method as claimed in claim 5 wherein the DNA thermodynamic parameters include dangling end parameters.

7. The method as claimed in claim 5 wherein the DNA thermodynamic parameters include coaxial stacking parameters.

8. The method as claimed in claim 5 wherein the DNA thermodynamic parameters include terminal mismatch parameters.

9. The method as claimed in claim 1 wherein the thermodynamic parameters include RNA thermodynamic parameters.

10. The method as claimed in claim 1 wherein the thermodynamic parameters include hybrid DNA/RNA thermodynamic parameters.

11. The method as claimed in claim 1 wherein the thermodynamic parameters include DNA loop thermodynamic parameters.

12. The method as claimed in claim 1 wherein the hybridization information represents top and bottom strand sequences which form a duplex and wherein the hybridization thermodynamics are calculated for the duplex.

13. The method as claimed in claim 1 wherein the hybridization information represents at least a section of a target and a length of at least one primer or probe complimentary to the target.

14. The method as claimed in claim 13 wherein the hybridization thermodynamics are calculated for a plurality of primers or probes complimentary to the target.

15. The method as claimed in claim 1 wherein the hybridization information represents at least a section of a target and a primer or probe.

16. The method as claimed in claim 15 wherein a length of the target is longer than a length of the primer or probe and wherein the hybridization thermodynamics are calculated for a best target/primer or target/probe complex and for competitive mismatch complexes.

17. The method as claimed in claim 14 wherein hybridization information represents at least a section of a target and a primer or probe and wherein a length of a target is longer than the length of the primer or probe and wherein the hybridization thermodynamics are calculated for a best target/primer or target/probe complex and for competitive target/primer or target/probe complexes.

18. The method as claimed in claim 2 further comprising, calculating concentration of each species in a solution at a plurality of temperatures.

19. The method as claimed in claim 18 wherein hybridization information also represents a primer or probe and wherein the length of the target is longer than a length of the primer or probe and wherein the hybridization thermodynamics are calculated for a best target/primer or target/probe complex and for competitive mismatch complexes and wherein the method further comprises calculating concentration of every species in a solution at a plurality of temperatures.

20. The method as claimed in claim 19 wherein the hybridization thermodynamics are calculated for at least two best target/primer or target/probe complexes and for their corresponding competitive mismatch complexes and wherein the method further comprises correcting for any interactions between the at least two best target/primer or target/probe complexes and their components.

21. A system for predicting nucleic acid hybridization thermodynamics, the system comprising:
a database of thermodynamics parameters;
means for receiving hybridization information which represents at least one sequence;
means for receiving correction data;
receiving a first set of data which represents hybridization conditions; and
means for calculating hybridization thermodynamics including net hybridization thermodynamics based on the hybridization information, the thermodynamic parameters, the correction data and the first set of data.

22. The system as claimed in claim 21 wherein the hybridization thermodynamics of individual single stranded, bimolecular and higher order complexes are statistically weighted in a numerical process and the equilibrium concentration of each species is output.

23. The system as claimed in claim 22 wherein the correction data includes folding correction data.

24. The system as claimed in claim 22 wherein the correction data includes linear correction data.

25. The system as claimed in claim 21 wherein the thermodynamic parameters include DNA thermodynamic parameters.

26. The system as claimed in claim 25 wherein the DNA thermodynamic parameters include dangling end parameters.

27. The system as claimed in claim 25 wherein the DNA thermodynamic parameters include coaxial stacking parameters.

28. The system as claimed in claim 25 wherein the DNA thermodynamic parameters include terminal mismatch parameters.

29. The system as claimed in claim 21 wherein the thermodynamic parameters include RNA thermodynamic parameters.

30. The system as claimed in claim 21 wherein the thermodynamic parameters include hybrid DNA/RNA thermodynamic parameters.

31. The system as claimed in claim 21 wherein the thermodynamic parameters include DNA loop thermodynamic parameters.

32. The system as claimed in claim 21 wherein the hybridization information represents top and bottom strand sequences which form a duplex and wherein the hybridization thermodynamics are calculated for the duplex.

33. The system as claimed in claim 21 wherein the hybridization information represents at least a section of a target and a length of at least one primer or probe complimentary to the target.

34. The system as claimed in claim 33 wherein the hybridization thermodynamics are calculated for a plurality of primers or probes complimentary to the target.

35. The system as claimed in claim 21 wherein the hybridization information represents at least a section of a target and a primer or probe.

36. The system as claimed in claim 35 wherein a length of the target is longer than a length of the primer or probe and wherein the hybridization thermodynamics are calculated for a best target/primer or target/probe complex and for competitive mismatch complexes.

37. The system as claimed in claim 34 wherein hybridization information represents at least a section of a target and a primer or probe and wherein a length of a target is longer than the length of the primer or probe and wherein the hybridization thermodynamics are calculated for a best target/primer or target/probe complex and for competitive target/primer or target/probe complexes.

38. The system as claimed in claim 22 further comprising means for calculating concentration of each species in a solution at a plurality of temperatures.

39. The system as claimed in claim 38 wherein hybridization information also represents a primer or probe and wherein the length of the target is longer than a length of the primer or probe and wherein the hybridization thermodynamics are calculated for a best target/primer or target/probe complex and for competitive mismatch complexes and wherein the system further comprises means for calculating concentration of every species in a solution at a plurality of temperatures.

40. The system as claimed in claim 39 wherein the hybridization thermodynamics are calculated for at least two best target/primer or target/probe complexes and for their corresponding competitive mismatch complexes and wherein the system further comprises means for correcting for any interactions between the at least two best target/primer or target/probe complexes and their components.

41. A computer-readable storage medium having stored therein a database of thermodynamics parameters and a computer program which executes the steps of:
receiving hybridization information which represents at least one sequence;
receiving correction data;
receiving a first set of data which represents hybridization conditions; and
calculating hybridization thermodynamics including net hybridization thermodynamics based on the hybridization information, the thermodynamic parameters, the correction data and the first set of data.

42. The storage medium as claimed in claim 41 wherein the hybridization thermodynamics of individual single stranded, bimolecular and higher order complexes are statistically weighted in a numerical process and the equilibrium concentration of each species is output.

43. The storage medium as claimed in claim 42 wherein the correction data includes folding correction data.

44. The storage medium as claimed in claim 42 wherein the correction data includes linear correction data.

45. The storage medium as claimed in claim 41 wherein the thermodynamic parameters include DNA thermodynamic parameters.

46. The storage medium as claimed in claim 45 wherein the DNA thermodynamic parameters include dangling end parameters.

47. The storage medium as claimed in claim 45 wherein the DNA thermodynamic parameters include coaxial stacking parameters.

48. The storage medium as claimed in claim 41 wherein the DNA thermodynamic parameters include terminal mismatch parameters.

49. The storage medium as claimed in claim 41 wherein the thermodynamic parameters include RNA thermodynamic parameters.

50. The storage medium as claimed in claim 41 wherein the thermodynamic parameters include hybrid DNA/RNA thermodynamic parameters.

51. The storage medium as claimed in claim 41 wherein the thermodynamic parameters include DNA loop thermodynamic parameters.

52. The storage medium as claimed in claim 41 wherein the hybridization information represents top and bottom strand sequences which form a duplex and wherein the hybridization thermodynamics are calculated for the duplex.

53. The storage medium as claimed in claim 41 wherein the hybridization information represents at least a section of a target and a length of at least one primer or probe complimentary to the target.

54. The storage medium as claimed in claim 53 wherein the hybridization thermodynamics are calculated for a plurality of primers or probes complimentary to the target.

55. The storage medium as claimed in claim 41 wherein the hybridization information represents at least a section of a target and a primer or probe.

56. The storage medium as claimed in claim 55 wherein a length of the target is longer than a length of the primer or probe and wherein the hybridization thermodynamics are calculated for a best target/primer or target/probe complex and for competitive mismatch complexes.

57. The storage medium as claimed in claim 54 wherein hybridization information represents at least a section of a target and a primer or probe and wherein a length of a target is longer than the length of the primer or probe and wherein the hybridization thermodynamics are calculated for a best target/primer or target/probe complex and for competitive target/primer or target/probe complexes.

58. The storage medium as claimed in claim 42 wherein the program further executes the step of calculating concentration of each species in a solution at a plurality of temperatures.

59. The storage medium as claimed in claim 58 wherein hybridization information also represents a primer or probe and wherein the length of the target is longer than a length of the primer or probe and wherein the hybridization thermodynamics are calculated for a best target/primer or target/probe complex and for competitive mismatch complexes and wherein the program executes the step of calculating concentration of every species in a solution at a plurality of temperatures.

60. The storage medium as claimed in claim 59 wherein the hybridization thermodynamics are calculated for at least two best target/primer or target/probe complexes and for their corresponding competitive mismatch complexes and wherein the program executes the step of correcting for any interactions between the at least two best target/primer or target/probe complexes and their components.
